(19) United States
(12) Patent Application Publication
(10) Pub. No.: US 2010/0233778 A1
(43) Pub. Date: Sep. 16, 2010

(54) DEVICE AND PROCESS FOR GENERATING BIOGAS FROM ORGANIC MATERIALS

(75) Inventor: Günter Perske, Mainhardt (DE)

Correspondence Address: DOBRUSIN & THENNISCH PC, 29 W LAWRENCE ST, SUITE 210, PONTIAC, MI, 48342, US

(73) Assignee: ELTAGA LICENSING GMBH, Muenchen (DE)

(21) Appl. No.: 12/161,714

(22) PCT Filed: Feb. 2, 2007
(86) PCT No.: PCT/DE2007/000193

§ 371 Date: Jun. 1, 2010

(30) Foreign Application Priority Data
Feb. 3, 2006 (DE) 10 2006 005 066.5

(51) Int. Cl. C12P 1/04; C12M 1/02

(52) U.S. Cl. 435/170; 435/289.1

(57) ABSTRACT

The invention relates to a device for generating biogas from organic materials having a biogas reactor which has a charging chamber for being charged with the organic materials and a backflow channel for an at least partial discharge of the organic materials from the biogas reactor. According to the invention it is provided that the biogas reactor in addition has at least one intermediate chamber, the charging chambers which form at least one intermediate chamber and the backflow channel form in this sequence sections of a flow path through which flow can pass in only one direction for the organic materials, two sequentially following sections respectively forming a rising flow path in one case and a falling flow path in the other. In addition the invention relates to a process for biogas generation.

The invention relates to a device for generating biogas from organic materials, said device comprising a biogas re-actor comprising a charging chamber for being charged with the organic materials and a backflow channel for at least partially discharging the organic materials from the biogas reactor.

The invention further relates to a process for generating biogas.

During the generation of biogas anaerobic bacteria are used to decompose organic materials no longer connected to the living organism and to convert them into a gas. Anaerobic bacteria are the last interlink in the in the natural cycle and occur everywhere in nature, for example in the stomachs of ruminants or in the black mud of lakes and moors. In the anaerobic fermentation first the facultative methylotrophs are to be differentiated from the obligate methylotrophs. The organic materials serving as raw materials in the anaerobic fermentation comprise, for example, organic materials or residues from industry, gastronomy, trade, agriculture (slurry and solid dung) or regrowing raw products (corn silage, grass silage and other short plants). These organic materials mainly consist of carbohydrates, fats and proteins. The random facultative methylotrophs can also live in the presence oxygen. They take over a first phase of the treatment and decompose the organic materials into alcohols, fatty acids and their salts. Said first phase of the treatment is referred to as acidogenic phase or hydrolysis. In a second phase the obligate methylotrophs adopt the transformation of the alcohols, fatty acids and their salts into a gas. This second phase is referred to as the methanation phase. The first and the second Phase occur delayed by approximately six hours, the so-called hydrolysis phase taking place during the first six hours.

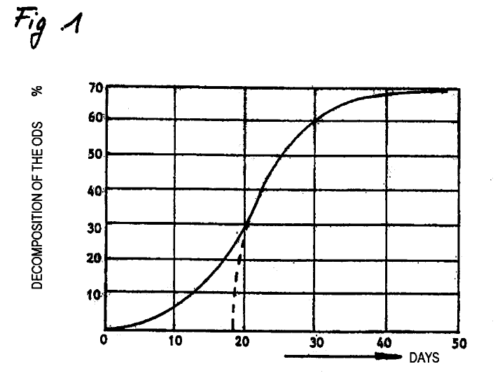

FIG. 1 shows a diagram generally illustrating the process of the natural fermentation. In particular the diagram shows the decomposition of an organic dry substance (ODS) in percent depending on the days elapsed (continuous line). In this connection it is to be observed that during the first twenty days the decomposition of the dry substance is launched very slowly. Only few bacteria, as present in all organic waste materials, will develop in the logarithmic ratio (broken line) in the presence of the corresponding nutrient supply. In the same ratio in which the bacteria develop the organic mass is broken down and transformed into a gas. A decomposition of the organic dry substance of 70 percent is desirable, however, this is, according to the diagram of FIG. 1, only achieved after approximately 40 days in the course of the natural fermentation process. The purpose of biogas systems is the provision of an environment for the organic fermentation enabling a significant acceleration of the organic fermentation.

Such a device and process for generating biogas are known from the DE 30 10 183 A1. The device comprises the features of the preambles of claims 1 and 13. The device for generating biogas, however, suffers from too low a percentage of achievable decomposition of the organic dry substance and thus of too low a gas yield.

It is therefore the object of the present invention to further develop the generic device for generating biogas and the process for generating biogas so that a higher gas yield can be obtained.

Said object is solved by the features of the independent claims.

Advantageous embodiments and further developments of the invention will become obvious from the dependent claims.

The device for generating biogas according to the invention is based on the generic state of the art in that the biogas reactor further comprises at least one intermediate chamber, the charging chamber, the at least one intermediate chamber and the backflow channel forming, in the named sequence, portions of a flow path for the organic materials which can only be passed in one direction, respectively one of the two successive portions forming a rising flow path and the other a falling flow path. With this measure it is achieved that the flow flows through the biogas reactor smoothly and uniformly in one direction. By providing at least one additional intermediate chamber the individual chambers are prevented from becoming too large since then there would be a risk that different flow zones are formed which detrimentally influence the natural fermentation process. In case of a varying consistency of the fermenting mass thus flow peaks of the supplied fermenting mass can be prevented from reaching the surface so that almost unfermented fermenting mass or fresh substrate leaks from the acidogenic phase at the outlet. With the subdivision into several chambers a plurality of fermentation chambers are provided inside of the biogas reactor whereby the flow is rendered more uniform and controllable. In this way the freshly supplied substrate has to pass the entire flow path before it can leave the biogas reactor, and it is therefore forced to pass the entire process in a predetermined period of time. With this measure the yield of biogas can be distinctively increased.

The device for generating biogas according to the invention may advantageously be further developed by providing the charging chamber with an overflow rim by means of which the at least one intermediate chamber can be filled with organic materials from the charging chamber. With said overflow rim a static inclination is formed at the transition between the charging chamber and the intermediate chamber where the fermenting mass may preferably degas. As long as the fermenting mass is moving forward while being contained in chambers the gases are tendentially rather enclosed. While the fermenting mass falls down over the overflow rim the fermenting mass is given the opportunity that the gases completely escape under a reduced resistance.

In a preferred embodiment of the device for generating biogas according to the invention it is further contemplated that a total of at least two intermediate chambers are provided, said intermediate chambers being connected in a section below a charging orifice of the backflow channel and above the overflow rim. With this measure the advantage mentioned above is made use of to an even more comprehensive extent. By subdividing the fermentation container into even more chambers the flow will become even more controllable, wherein, in particular, by connecting the two intermediate chambers, these are connected in accordance with the principle of communicating containers, and the fermenting mass has to subsequently flow through said two chambers.

In addition the device for generating biogas according to the invention may be further developed so that the intermediate chambers are formed in the interior of a cup-like interior reservoir disposed in the interior of the biogas reactor. In this way a concrete possibility of subdividing the biogas reactor into a plurality of chambers is given.

Furthermore the device for generating biogas according to the invention may be further developed so that a discharge channel branches from the backflow channel so that the discharge channel forms a communicating tube together with the backflow channel. In this way a certain portion of the fermenting mass from the biogas reactor is automatically discharged when new fermenting mass is supplied. In this connection no further pumps or valves optionally opening or closing the discharge channel are required inside of the biogas reactor.

In addition the device for generating biogas according to the invention may be formed so that the cross sectional area of the charging chamber is narrowed in the area of the overflow rim. Due to said narrowing in the upper section of the charging chamber the overflowing substrate is compressed and therefore counteracts the formation of floating layers. Preferably said narrowing of the cross section amounts to 50%. Floating layers would lead to an aggravation of a degassing of the fermenting mass. Further the fermenting mass is compressed in the narrowed zone, and in this way inviscid fermenting mass is prevented from flowing through or past viscous fermenting mass. Owing to the narrowing the internal frictional resistances in the substrate are increased so that no final mixture can be obtained whereby the formation of floating layers can be reliably prevented. The formation of floating layers would result in a tipping or even standstill of the fermentation process. Chiefly in the first third of the fermentation process most of the biogas is generated, and there is thus the greatest risk that a floating layer will form.

The same advantages can be achieved by providing the biogas reactor with an upper portion tapered towards the upper side and by having the overflow rim extend so far into the upper portion that the cross sectional area of the charging chamber is narrowed in the area of the overflow rim. In addition to the named advantages this has the effect that the friction is increased for the fermenting mass by the transition into the conic portion and inside of the conic portion itself which, as explained above, counteracts the formation of floating layers.

The device for generating biogas according to the invention may further be configured so that the biogas reactor comprises a lower portion tapered towards the lower side, a receptacle for sediments being formed in the lower section of the lower portion which is, on the one hand, optionally connectable to the interior of the biogas reactor, and, on the other hand, optionally connectable to the surroundings of the biogas reactor. Due to this measure sediments or impurities such as sand or other dense media will deposit toward the lower side in the interior of the biogas reactor and may be removed from there from time to time. Thus cloggings in the flow paths within the biogas reactor are excluded. Besides such sediments may affect the fermentation process. With such a clearable receptacle such an impairment is excluded. In the present process a fully fermented mass decomposed by more than 90% and containing hardly any organic matter any more is reintroduced into the process as an injected mass with a reintroduction rate of 50% in a cycle of approximately 10 days. The salts and other anorganic parts contained therein are to be removed via the receptacle after two 2 fermentation cycles at the latest.

The device for generating biogas according to the invention may, furthermore, be formed so that a gas discharge line via which a constant pressure can be applied to the interior of the biogas reactor is connected in the upper section of the biogas reactor. In this manner a measure to keep the pressure inside of the biogas reactor constant is provided which is easy to realise, low-priced and very reliable. Therefore expensive valves can be omitted which would wear down extremely rapidly when used in the environment of a biogas reactor.

Furthermore the device for generating biogas can be embodied so that further a liquid reservoir is provided which can be filled with a liquid, the end of the gas discharge line leading out of the interior of the biogas reactor being disposable in the liquid so that the pressure in the interior of the biogas reactor is adjustable via the immersion depth in the liquid. With this additional liquid reservoir the pressure inside of the biogas reactor can be varied whereby optimum fermentation conditions can be adjusted. At the same time said pressure adjustment by means of a liquid reservoir are very easy to realise and inexpensive. In addition possible pollutants are removed from the biogas discharged from the gas discharge line through the liquid.

The device for generating biogas according to the invention may, in addition, be further developed by further providing a mixing unit for charging the charging chamber which is adjusted so as to mix new organic materials and organic materials discharged from the biogas reactor by means of the recirculation conduit in a ratio of substantially 1:1. The organic materials discharged from the biogas reactor are, in this case, fully fermented and non-reactive substances. With a mixture in a ratio of 1:1 optimum results can be achieved. Since the mayor part of the organic materials is decomposed at the end of the fermentation processes the methylotrophs are present in the highest concentration. The mixture of these organic materials discharged from the biogas reactor and the newly introduced organic materials ensures that the fermentation process starts very turbulently and rapidly since the large number of bacteria is confronted with large organic masses and therefore causes the turbulently induced fermentation process.

Above that the device for generating biogas may be further developed by further providing a heat exchanger disposed upstream of the charging chamber and adjusted so as to preheat the organic materials to be newly supplied by means of a hot fluid. With this preheating a supply of the fermenting mass to the biogas reactor at a temperature optimum for the fermentation process is achieved. In this connection optimum temperature is to indicate a temperature at which the gas yield is maximum.

Furthermore the device for generating biogas can be formed so that a crushing unit is provided upstream of the charging chamber. By crushing the supplied organic materials the substances form an aqueous, pumpable mixture of a wide range of organic components, an ideal forage for the bacteria. By crushing a good thorough mixing, an enhanced pumpability and finally a more rapidly developing fermentation process are achieved. In this way the organic materials are homogenised, i.e. decomposed, so that the structure of the organic parts is mildly broken and the contained water is released. Thereby a large surface area is provided, and the bacteria can settle more intensively.

As a further development the device according to the invention can be configured so that a gasholder is provided in which biogas generated in the biogas reactor can be stored and which at least partly surrounds the biogas reactor. This further development is advantageous in that the gas container has an insulating effect on the biogas reactor so that the heat is better held in the biogas reactor and less heat energy has to be supplied to the biogas reactor. At the same time a container for storing biogas is provided by this further development without additional components.

The heat exchanger for a biogas system is based on the generic state of the art by a cleaning device which can be slipped over the heat introduction body when the heat exchanger housing is closed. The problem of a heat exchanger in connection with a viscous organic mass is that protein flocculates and deposits on the heat introduction bodies from approximately 60° C. The heat exchanger enables a permanent removability of such deposits to ensure a good passage and a good heat transfer. In addition a movable cleaning device provides for a good thorough mixing of the mass whereby it is uniformly heated.

Above that the heat exchanger may be further developed so that the cleaning device can be reciprocated by means of a spindle drive. With the spindle drive a movement of the cleaning device may be realised in a low-maintenance and cost-effective way.

In addition the heat exchanger may be configured so that the heat introduction body consists of double-walled pipes in which the feed line surrounds the return line or the return line surrounds the feed line. Owing to the use of double-walled pipes the feed line and the return line of the hot fluid can be lead out of the housing of the heat exchanger on the same side. In this way the heat exchanger can be more readily opened on the opposing side of the heat exchanger. In addition the hot fluid runs twice the distance through the double-walled pipes inside of the heat exchanger than it would if normal, single-walled pipes were provided.

With the process for generating biogas according to the invention the advantages described above are achieved in an analogous manner.

In an advantageous manner the process can be further developed so that the process steps taking place in the biogas reactor are executed without any active stirring of the organic materials. By avoiding a stirring of the organic materials a disturbance of the fermentation process is avoided. The hydrolysis phase is sensitive and will not tolerate any interference.

In addition the process may be further developed so that the newly supplied organic materials are preheated to 35° C.-37° C. by means of a heat exchanger before being introduced into the charging chamber. In this way the fermentation process takes place in the mesophilic range whereby higher decomposition rates and therefore a greater amount of gas can be obtained. Processes according to the state of the art frequently function in the thermophilic range (of approximately 55° C.) whereby a sanitisation and a more rapid progression of the process are achieved, however, more gas is not generated in this way. In the mesophilic range the energy balance is better since less energy is required for heating purposes.

Besides the process can be further developed so that the organic materials consisting of newly supplied, pre-heated organic materials and organic materials directly discharged by means of the backflow channel and introduced into the charging chamber are mixed. Owing to said mixing with organic materials which have already passed the biogas reactor the newly supplied organic materials are mixed with fully active bacteria so that the fermentation process can be started very quickly.

It may further be contemplated that the mixture ratio is 1:1 during the process. This mixture ratio has been found to be the optimum mixture ratio for a maximum gas yield.

Finally the process according to the invention can be further developed so that an internal pressure in the interior of the biogas reactor is kept constant. Due to the constant internal pressure the fermentation process is interfered with to the smallest possible extent which has a positive effect on the fermentation process. Due to the constant internal pressure uncontrolled flows of the fermenting mass are prevented.

A preferred embodiment of the invention will be described below with reference to the figures in which:

FIG. 1 is a diagram generally illustrating the progress of the natural fermentation;

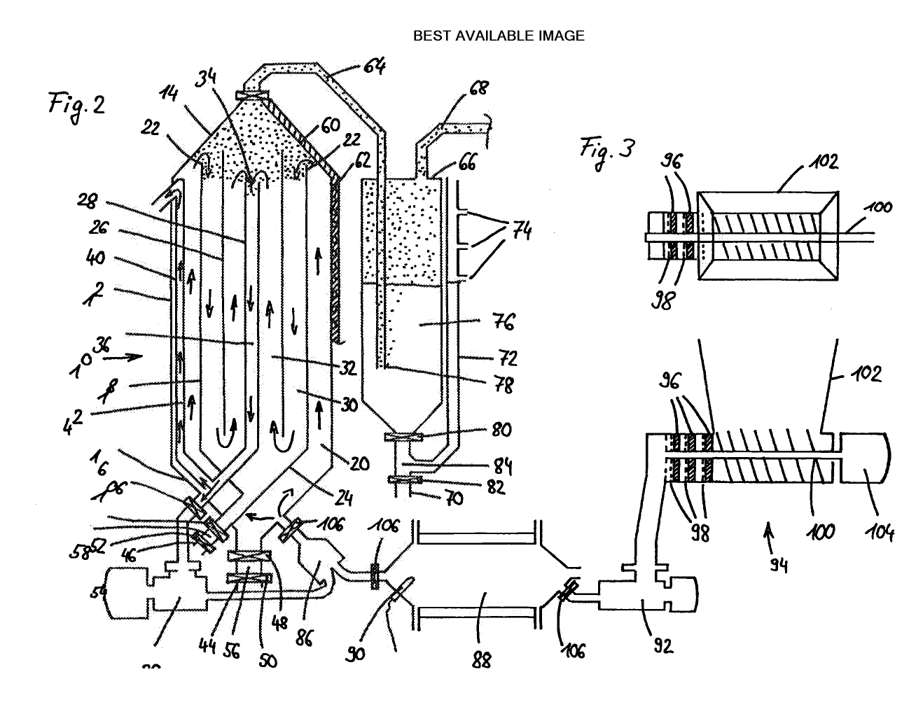

FIG. 2 is a schematic representation of the device for generating biogas according to the invention;

FIG. 3 is a plan view of a crushing unit of the device according to FIG. 2;

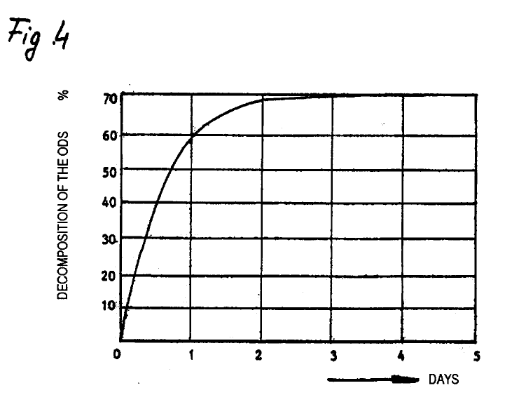

FIG. 4 shows a progress of the fermentation by which the process of the present invention is securely achieved;

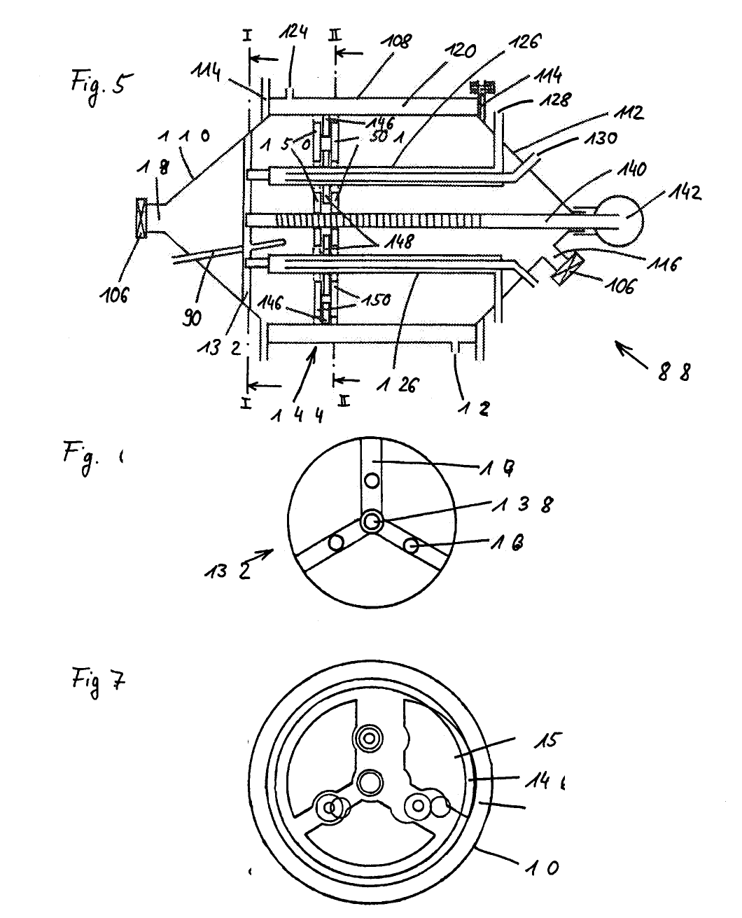

FIG. 5 is a schematic cross sectional view of a heat exchanger of the device according to FIG. 2;

FIG. 6 is a schematic cross sectional view along the line I-I in FIG. 5;

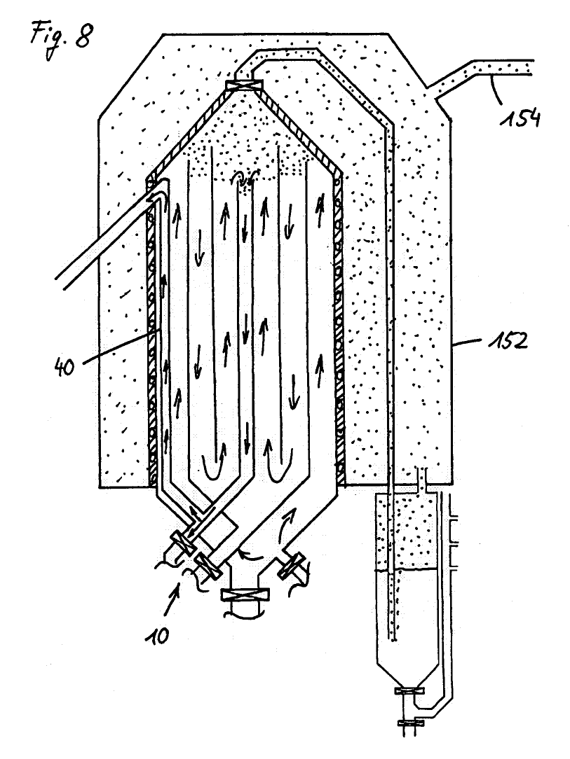

FIG. 7 is a schematic cross sectional view along the line II-II in FIG. 5; and

FIG. 8 is a schematic representation of a further development of the device for generating biogas according to the invention.

FIG. 2 shows a schematic representation of the device for generating biogas according to the invention. In the preferred embodiment the biogas reactor 10 comprises an external container 12 which is preferably cylindrical in a central portion and conically tapered towards the end in an upper portion 14 as well as in a lower portion 16, respectively. In the interior of the external container 12 an interior reservoir 18 is accommodated which is cup-shaped and arranged in a substantially constant distance to the external container 12 so that a charging chamber 20 surrounding the interior reservoir 18 is formed between the external container 12 and the interior reservoir 18. The external container 12 as well as the interior reservoir 18 are preferably formed of steel, however, an embodiment consisting of other materials such as, for example, plastic materials, can also be realised. An upper edge of the interior reservoir 18 serving as an overflow rim 22 in the present embodiment is inserted so far into the upper portion 14 tapered towards the upper side that the cross sectional area of the charging chamber 20 disposed inbetween narrows by approximately 50% towards the upper side. In this connection, however, it is only important that the cross section narrows towards the end of the charging chamber 20. Alternatively this may also be achieved in that the upper tip of the overflow rim 22 is bent to the outside, i.e. towards the exterior housing 12. It is also possible that the narrowing of the cross sectional area is not achieved by the interaction of the upper portion 14 tapered towards the upper side and the overflow rim 22 but that a separate guide plate is provided on the upper end of the charging chamber 20, said guide plate being attached to the exterior housing 12 and narrowing the cross section of the charging chamber 20 towards the upper side so that the required narrowing of the cross section is achieved. In the lower section the interior reservoir 18 is tapered towards the lower side in a similar manner as the external container 12. The interior reservoir 18 is preferably cylindrical and conical in the tapered lower portion 24. Inside of the interior reservoir 18 a cylindrical inner pipe 26 is disposed so that substantially the same distance is obtained between the inner pipe 26 and the interior reservoir 18 as between the interior reservoir 18 and the external container 12. The lower edge of the inner pipe 26 extends downwards almost as far as the cylindrical portion (the portion which is not tapered) of the interior reservoir 18. The upper edge of the inner pipe 26 extends further upwards than the overflow rim 22. In the interior of the inner pipe 26 a backflow pipe 28 is provided which extends downwards into the portion 24 of the interior reservoir 18 where the backflow pipe 28 is lead out of the interior reservoir 18 inside of the inner pipe 26. The backflow pipe 28 extends so far upwards that the upper edge of the backflow pipe 28 is, with respect to the vertical plane, disposed below the overflow rim 22. Advantageously the upper edge of the backflow pipe 28 extends substantially as far upwards as the central (preferably cylindrical) portion of the external containers 12. In the present embodiment the external container 12, the interior reservoir 18, the inner pipe 26 and the backflow pipe 28 are disposed concentrically. Between the outer side of the inner pipe 26 and the inner side of the interior reservoir 18 a first, substantially cylindrical intermediate chamber 30 is formed. Between the outer side of the return pipe 28 and the inner side of the inner pipe 26 a second, substantially cylindrical intermediate chamber 32 is formed. The first intermediate chamber 30 and the second intermediate chamber 32 are connected to each other in the lower portion. The upper edge of the return pipe 28 forms a charging orifice 34. In the interior of the return pipe 28 a backflow channel 36 is formed. As mentioned above, the backflow pipe 28 is lead out of the interior reservoir 18 into the lower portion 24 of the interior reservoir 18, passes the wall of the external container 12 in the lower portion 16 and is lead into an injection pump 38 which is preferably a helical rotor pump. On the portion of the return pipe 28 extending inside of the charging chamber 20 a discharge pipe 40 branches off which extends so far upwards into the charging chamber 20 that an upper orifice of the discharge pipes 40 is located in approximately the same height as the charging orifice 34 of the return pipe 28. In the upper section the discharge pipe 40 is formed so that the upper portion of the pipes is bent by more than 90 degrees and that the bent portion extends to the outside through the wall of the external container 12. The discharge channel 42 formed by the discharge pipe 40 is thus U-shaped and connected to the backflow channel 36 so that the backflow channel 36 and the discharge channel 42 form a communicating pipe. The charging chamber 20 is formed so that it can be charged with organic materials or an organic substance from the outside in a lower section. The organic substance is also fed through the charging chamber 20, the first intermediate chamber 30 and the second intermediate chamber 32 in a manner described in more detail later, wherein the organic substance may still contain sediments or dense media. Therefore a respective pipe section 44 and 46 branches off at the bottom end of the external container 12 and of the interior reservoir 18, said pipe sections 44 and 46 being provided with a slider 48, 52 in the vicinity of the respective container and with another slider 50, 54 in a certain distance thereto. Using the respective sliders the respective pipe section 44, 46 may be selectively opened and closed. The distance between the slider 50 and the slider 48 is preferably approximately 80 cm, and the distance between the slider 52 and the slider 54 is preferably 60 cm. During the normal operation the sliders 48 and 52 are opened, and the sliders 50 and 54 are closed. Therefore, when the sediments contained in the organic substrate sink downwards they slide along the portions 16 and 24 towards the centre of the respective container 12, 18 and leave it through the respective opened slider 48, 52 in the respective pipe section 44, 46. There the sediments are collected at the closed sliders 50, 54. The pipe section between the sliders 48 and 50 as well as the pipe section between the sliders 52 and 54 thus respectively form a receptacle 56, 58 for sediments. Preferably the pipe sections are formed so as to be transparent at the collecting chambers 56, 58, for example by means of acryl glass, so that the amount of collected sediments can be monitored. When a certain amount is reached the collected sediments can be removed by closing the respective sliders 48 and 52 to prevent the containers 12, 18 from leaking. Then the respective sliders 50 and 54 are opened and the collecting chambers 56, 58 are emptied. For the normal operation the sliders 50 and 54 are closed again, and the sliders 48 and 52 are opened. An insulation 60 of which only portions are shown completely surrounds the external container 12 (the supply and discharge lines are left blank) so that the temperature of preferably 35° C. which is advantageous for the generation of biogas can be kept as constant as possible in the interior of the biogas reactor 10 and that less energy has to be supplied to maintain said temperature. A heater 62 is embedded in the insulation 60, said heater being embodied in the form of helically arranged water pipes containing water which is, for example, heated in a combined heat and power unit (not shown) in the preferred embodiment. Alternatively heating wires may also be embedded in the insulation 60. Preferably the heater 62 surrounds the external container 12 from the lower side up to below the upper portion 14. For protecting the insulation 60 the insulation 60 including the heater 62 may be surrounded by a protective jacket like, for example, a metal sheet jacket.

On the upper end of the external container 12 which is the tapered end of the upper portion 14 a gas discharge line 64 branches off. Said gas discharge line 64 is lead downwards outside of the external container 12 and next to it, an end portion of the gas discharge line 64 penetrating a liquid reservoir 66 and extending downwards inside of said liquid reservoir 66. The liquid reservoir 66 is preferably a cylindrical container the lower portion of which is conically tapered towards the lower side. At the upper side of the liquid reservoir 66 a gas supply line 68 branches off via which the gained biogas is supplied to a gasholder (not shown) from where it is available for a combined heat and power unit (not shown) for generating power. On the lower end of the liquid reservoir 66 a pipe section 70 is lead out of the liquid reservoir 66. A riser pipe 72 branches off said pipe section 70, the riser pipe 72 being guided upwards up to the upper edge of the liquid reservoirs 66 next to the liquid reservoir 66. The riser pipe 72 is open on the upper side, and between the upper edge of the riser pipe 72 and more than 1 m below the upper edge three orifices 74 are formed, the lowest of the orifices being located more than 1 m below the upper edge of the riser pipe 72. The distance between the lowest of the three orifices 74 and the uppermost of the three orifices 74 is preferably 1 m. The riser pipe 72 is connected to the interior of the liquid reservoir 66 in accordance with the principle of communicating pipes. During the operation the interior of the liquid reservoir 66 is filled with a liquid 76, preferably water, the liquid level of which is adjustable by means of the orifices 74. Due to the principle of the communicating pipes the same liquid level is prevailing in the riser pipe 72 as well as in the liquid reservoir 66 so that, if the lowest of the orifices 74 is opened, the liquid reservoir 66 can be filled with liquid 76 up to a level corresponding to the level of the lowest of the orifices 74. If the lowest of the orifices 74 is closed, for example by means of a plug, the liquid reservoir 66 can be filled up to a higher liquid level corresponding to the level of the orifice 74 located further upwards. If all orifices 74 are closed the liquid reservoir 66 can be completely filled, the liquid reaching the upper edge of the riser pipe 72 when the complete charge is reached. The end 78 of the gas discharge line 64 leading out of the external container 12 is disposed inside of the liquid reservoir 66 so that said end 78 is immerged in the liquid 76. The lower orifice of the end 78 is spaced apart from the uppermost one of the orifices 74 of the riser pipe 72 by 2 m. The immersion depth of the gas discharge line 64 in the liquid 76 thus amounts to a minimum of 1 m when the lowest of the three orifices 74 is opened, and to a maximum of 2 m if only the uppermost one of the three orifices 74 is opened. Due to this adjustable immersion depth of the end 78 of the gas discharge line 64 the pressure inside of the external container 12 can be adjusted to a constant value. When charged with water therefore a pressure of 0.1 bar is obtained in the external container 12 with an immersion depth of 1 m. With an immersion depth of 2 m a pressure of 0.2 bar is adjusted in the external container 12. As described above, the pipe section 70 is lead out at the lower end of the liquid reservoir 66. Here a slider 80 is provided in the pipe section between the outlet at the liquid reservoir 66 and the branch of the riser pipe 72, and a slider 82 is provided in the pipe section behind the branch of the riser pipe 72. With these two sliders 80, 82 the passage through the pipe section 70 may selectively be opened or closed. During the normal operation the slider 80 is opened, and the slider 82 is closed, whereby a receptacle 84 for sediments is formed. Thus impurities contained in the biogas are filtered out by the liquid 76. The gas rises upwards in the liquid 76, and the sediments filtered out sink down-wards in the liquid 76, are then guided to the centre by the tapered shape of the lower portion of the liquid reservoir 66 and collected in the receptacle 84. In the area of the receptacle 84 the pipe section 70 may be transparent, for example, formed of acryl glass, so that the collection of sediments can be monitored. When the amount of sediments in the receptacle 84 has reached a certain quantity the slider 80 can be closed, and the slider 82 can be opened so that the sediments can be discharged from the system at the lower end of the pipe section 70. After having emptied the receptacle 84 the slider 82 is closed, and the slider 80 is opened again.

As mentioned above, the charging chamber 20 can be charged from below. To this end a pipe section connecting the charging chamber 20 to the outlet of a mixing unit 86 extends through the wall of the external container 12 in the lower portion 16. The outlet of the mixing unit 86 is tapered towards the charging chamber 20, preferably by 50%.

The inlets of the mixing unit 86 are connected to pipes by means of which the mixing unit 86 is connected to the outlets of the injection pump 38 and of a heat exchanger 88. The mixing unit 86 mixes organic materials supplied from the injection pump 38 and from the heat exchanger 88, preferably in a ratio of 1:1. Alternatively the device for generating biogas may also be operated with other mixture ratios. The heat exchanger 88 to be explained in more detail later comprises a temperature sensor 90 disposed close to the outlet by means of which the temperature of the organic substance disposed in the heat exchanger can be detected. The heat exchanger 88 is connected to a fresh substrate pump 92 which is preferably a helical rotor pump disposed on the inlet side. Said fresh substrate pump 92 in turn is connected to a crushing unit 94 arranged on the inlet side. The connection between the crushing unit 94 and the fresh substrate pump 92 as well as the connection between the fresh substrate pump 92 and the heat exchanger 88 is realised by means of pipe connections. The crushing unit 94 comprises three subsequently arranged cutting tools 96 such as, for example, star-shaped cutting knifes on the sides of which opposing the substrate flow a respective bushing 98 is disposed. The cutting tools 96 are arranged on a shaft 100. The shaft 100 is, at least in sections, formed as a worm conveyor shaft. For connecting the shaft 100 to the cutting tools 96 the shaft 100 is flattened in the area of the cutting tools 96 so that the cutting tools 96 can be accurately fitted on. The bushings 98 comprise a central round hole through which the shaft 100 is passed so that the bushings 98 do not interfere with the rotation of the shaft 100. The bushings 98 are locked by a bolt (not shown) so that the cutting tool 96 exerts a cutting and shearing effect on the supplied, possibly fibrous organic fresh substrate on the respective bushing 98. The dimensions of the holes of the bushings 98 are stepped so that the crushing is carried out stepwise and can be individually adjusted. Thus organic fresh substrate supplied via a hopper 102 is supplied to the cutting tools 96 by means of the shaft 100 formed as a worm conveyor, crushed by said cutting tools 96, and fed on to the fresh substrate pump 92. Here the shaft 100 and thus the cutting tools 96 are driven by a drive unit 104 (for example an electric motor). A plan view of the crushing unit is shown in FIG. 3.

Between the fresh substrate pump 92 and the heat exchanger 88, between the heat exchanger 88 and the mixing unit 86, between the mixing unit 86 and the external container 12, between the injection pump 38 and the external container 12 as well as between the external container 12 and the liquid reservoir 66 a respective slider 106 is disposed by which the respective pipe connections can be selectively opened and closed. During the normal operation all of these sliders 106 are opened, however, it may, for example for maintenance purposes, be required to close the respective sliders 106 upstream and/or downstream of a component when exchanging the component to enable an exchange of the component without organic materials leaking from the system.

The operation of the device for generating biogas according to FIG. 2 or a process for generating biogas using the device according to FIG. 2 will be described below. In the process according to the state of the art too little consideration was given to the fact that the two kinds of methylotrophs, the facultative methylotrophs and the obligate methylotrophs, live in a symbiosis, i.e. that they complement each other and depend on each other. The first phase described in the introduction (hydrolysis or acidogenic phase) and the second phase (methanation phase) of the fermentation processes take place delayed by approximately six hours, the so-called hydrolysis phase taking place during the first six hours. The prepared alcohols and fatty acids have to be processable in the following second phase as well. In this connection it is important that no interferences by stirring or mixing disturb the balance of the first phase by a renewed, excessive acid formation like in the process according to the invention. Any addition of fresh substrate will activate the acid formation so that an accumulation of acid products will be caused; if the processing of the organic materials does not function optimally the actual fermentation process will surrender to the excessive production of acid products. Therefore the acidogenic phase (hydrolysis) was intentionally given priority in the present process whereby the present process differs from many processes according to the state of the art. In the present process hydrolysis and methanation are balanced and not carried out separately like in the state of the art. If the hydrolysis and the methanation were carried out separately a pre-acidified substrate would have to be introduced into the active process which would lead to the formation of acid concentrations and to the process requiring a long period of time, sometimes of 20 to 30 days and more, for the establishment of an equilibrium. The same disadvantage will be caused by stirring and mixing as well as by an injection of gas. In the present process disturbances of this equilibrium by stirring and mixing are intentionally avoided.

Before the hopper 102 of the crushing unit 94 is charged with the organic materials it is advantageous to possibly sort the organic materials, i.e. to remove coarse perturbing materials and to crush larger parts to approximately 30 mm. The organic materials are then filled into the hopper 102. Then the drive unit 104 is manually or automatically turned on (for example by means of a light barrier (not shown), etc.). In this way the shaft 100 rotates so that the worm conveyor of the shaft 100 conveys the charged substrate to the first of the three cutting tools 96. The cutting tool 96 crushes the organic fresh substrate and conveys it through the corresponding bushing 98. Thereafter the organic fresh substrate passes the second and the third crushing stage consisting of the cutting tool 96 and the associated bushing 98 (according to FIG. 2 from the right side to the left side). With said crushing the organic materials from gastronomy, from grease separators, from the food industry and from other sources are homogenised, i.e. decomposed so that the structure of the organic parts is mildly broken up and that the trapped water is released. In this way a large surface is created, and the bacteria involved in the fermentation process can settle more intensively. The substrate thus obtained by means of the cutting tools 96 is an aqueous, pumpable mixture of a wide range of organic components. The feeding effect of the shaft 100 and the feeding effect of the cutting tools 96 transport the organic fresh substrate on to the fresh substrate pump 92. Said fresh substrate pump 92 is controlled by means of a controller (not shown) and pumps the fresh substrate into the heat exchanger 88 and from there on into the mixing unit 86 and finally into the charging chamber 20 of the biogas reactor 10. In this connection the fresh substrate supplied from the crushing unit 94 is first fed into the interior of the heat exchanger 88. Then the fresh substrate pump 92 is turned off. The fresh substrate fed into the heat exchanger 88 is heated, according to the present embodiment to 37° C., which is monitored by means of the temperature sensor 90. Said heating is achieved by introducing hot fluid spatially separated from the fresh substrate into the heat introduction bodies to be explained later on. The fluid preferably has a temperature of approximately 80° C. As soon as the temperature sensor 90 detects that the temperature of 37° C. is reached the fresh substrate pump 92 is turned on so that new fresh substrate is introduced into the heat exchanger 88 and the preheated fresh substrate is discharged from the heat exchanger 88 and introduced into the mixing unit 86. Together with the fresh substrate pump 92 simultaneously the injection pump 38 is operated as well which will be explained in more detail later. The fresh substrate pump 92 preferably remains turned on until the temperature sensor 90 detects a temperature of 35° C. or less. Then the fresh substrate pump 92 is turned off so that the organic fresh substrate newly introduced into the heat exchanger 88 which is not yet preheated can now be preheated until it reaches a temperature of 37° C. and is moved on as described above. The intervals in which the organic fresh substrate is supplied can be varied, and the fresh substrate pump 92 does not have to be controlled exclusively depending on the temperature sensor 90. The control by means of the temperature sensor 90 is rather to be understood so that it is a prerequisite for the supply of the fresh substrate that it has a minimum temperature of 35° C. The intervals may also be longer, as required for the preheating of the fresh substrate in the heat exchanger 88. Therefore an almost continuous supply or a supply of fresh substrate in certain cycles can be effected in case of a rapid pre-heating of the fresh substrate in the heat exchanger 88. The injection pump 38 serves to introduce substrate discharged from the biogas reactor 10 and also having a temperature of 35° C. into the mixing unit 86. As mentioned above the fresh substrate pump 92 and the injection pump 38 are operated synchronously so that respectively identical portions of a substrate are introduced into the mixing unit 86 by the injection pump 38 and the fresh substrate pump 92. It has been found that by mixing substrate discharged from the biogas reactor (non-reactive fermenting mass) and fresh substrate (substrate discharged from the heat exchanger 88) in a ratio of 1:1 the best results are achieved, provided that both mass flows have approximately the same temperatures. A tolerance of 1-2° C. is, however, acceptable. By mixing the non-reactive fermenting mass with fresh substrate in the correct ratio the fermentation process is boosted so that finally the remaining organic materials are attacked and decomposed. This measure considerably contributes to the high decomposition rate of the organic materials of the present method which may amount to 70% and more. In this connection it has to be noted that the mixing of the two mass flows may, in no case, be effected in an upstream container such as, for example, an upstream pit, otherwise gas will be lost.

In the mixing unit 86 the supplied substrate is mixed more thoroughly by the conically tapered outlet of the mixing unit 86. At the end of a fermentation process the major part of the organic materials is decomposed, and the methylotrophs are present in the highest concentration. The introduction of the methylotrophs in the fresh substrate by means of the mixing unit 86 ensures that the fermentation process starts violently since the large number of bacteria is confronted with a large quantity of fresh substrate and thus causes the violently initiated fermentation process.

After having entered the charging chamber 20 the substrate, driven by the following substrate, rises upwards in the charging chamber 20. The substrate flowing upwards in the charging chamber 20 is compressed when reaching the narrow portion in the upper area of the charging chamber 20 so that no floating layers will develop. The fermenting mass overflowing at the overflow rim 22 falls into the first intermediate chamber 30 via the overflow rim 22. During this drop over the overflow rim 22 the fermenting mass is completely degassed. In addition possible agglomerations of the substrate are broken up which enables and supports degassing. The falling height at this overflow rim 22 is, in practice, approximately 0.6 m and sometimes depends on the pressure applied to the interior of the biogas reactor 10. The biogas is collected in the upper portion 14 of the external container 12 as shown by dots in FIG. 2. Since the upper edge of the inner pipe 26 is higher than the overflow rim 22 a movement of the fermenting mass directly from the charging chamber 20 into the second intermediate chamber 32 is avoided. The fermenting mass introduced into the first intermediate chamber 30 rather moves downwards in said chamber. This downwards movement of the fermenting mass is driven by the replenished fermenting mass. The first intermediate chamber 30 is connected to the second intermediate chamber 32 in the lower section of the interior reservoir 18 so that the fermenting mass discharged at the lower end of the first intermediate chamber 30 is introduced into the lower end of the second intermediate chamber 32. In the second intermediate chamber 32 the fermenting mass rises upwards. Due to the principle of communicating containers substantially the same filling level prevails in the first intermediate chamber 30 and in the second intermediate chamber 32. This filling level corresponds to the charging orifice 34. When the fermenting mass reaches the charging orifice 34 in the second intermediate chamber 32 the fermenting mass falls into the backflow channel 36 and sinks downwards in it. The height by which the substrate falls downwards in the backflow channel 36 sometimes depends on the pressure applied to the interior of the biogas reactor 10. When falling from the second intermediate chamber 32 into the backflow channel 36 the biomass is also completely degassed. Due to the fact that the discharge channel 42 forms a communicating pipe together with the backflow channel 36 the filling level in the discharge channel 42 depends on the filling level in the backflow channel 36. About half of the substrate moving downwards in the backflow channel 36 leaves the biogas reactor via the discharge channel 42, and the other half is fed into the mixing unit 86 by the injection pump 38 where it is mixed with the newly supplied fresh substrate as described above. Inside of the external container 12 the biomass is maintained at a temperature of approximately 35° C. by the heater 62. The present process takes place in the mesophilic range (30° C.-38° C.) since in this range the decomposition rates are higher and therefore a larger amount of gas can be generated. The methylotrophs present in the process are very sensitive and require a temperature which is as uniform as possible and not subject to strong variations.

The backflow channel 36 is reached after approximately 8 to 10 days, the substrate only containing non-reactive fermenting mass in this position which is highly enriched with the prevailing fermentation bacteria. The biogas accumulating in the upper portion 14 of the external container 12 is maintained at a constant pressure by means of the liquid reservoir 66 and first continuously discharged into the liquid reservoir 66 via the gas discharge line 64 and from there introduced into a gasholder (not shown) via the gas supply line 68. Here no pressure valves are used which would easily wear down, the pressure is rather kept constant via the immersion depth of 1 m to 2 m. The delivery of the fermenting mass in the entire system is effected by means of the pump pressure of the pumps 38 and 92, by the hydrostatic inclination from the overflow rim 22 towards the charging orifice 34 and by the gas pressure of 0.1 to 0.2 bar in the upper portion 14. The feeding effect for the organic materials present in the biogas reactor can be varied using the static inclination as well as the internal pressure, i.e. the static inclination and/or the internal pressure can be adjusted to the consistency of the organic materials.

As a result a fermentation process is achieved in which the organic components are reduced by up to 70% or more in a relatively short period of time of approximately 6 to 10 days. That means the extraction of a large amount of gas and, in case of corresponding basic conditions, a very good efficiency. The transformation of the organic materials takes place in closed containers so that no odours escape to the outside. The fermentation residues discharged at the discharge pipe 40 have an earthy smell and are free of slime substances and compatible with the environment in every respect. They can be brought out on agricultural surfaces as natural fertiliser without any further treatment. Should a further treatment be required for mandatory reasons a solid-liquid separation is indicated and possible with little effort since the fully fermented residues can be readily separated.

The obtained amount of biogas is, with approximately 2 m³ biogas per kilograms of decomposed organic dry substance, within the achievable range. The biogas has a methane content of about 70-74% whereby an energy value of the biogas of 7.0-7.4 kWh/m³ is obtained. A further advantage of the device described above or the related method not to be underestimated is that substrates with a dry substance content of 20-25% can be fermented so as to be pulpy but still pumpable. Even in case of extremely high dry substance contents there are no flow-related problems in the biogas reactor 10 since the mixture of non-reactive fermenting mass and fresh substrate is diluted by the non-reactive fermenting mass when mixed before being introduced into the fermentation container, the substrate being enhanced to a pH value of approximately 7.5 to 7.8 which is preferred for a biocenosis. The device described above and the process described above ensure a decomposition of the organic materials of more than 90%. Thus an optimum fermentation is achieved, and the residues (the injected mass) have a pH value of more than 8.0 to 8.4. The result is that the reintroduction rate (the amount of the injected mass) has a direct influence on the substrate. A thorough mixture of fresh substrate and injected mass in the correct ratio, predominantly in a ratio of 1:1, constitutes the fermentation substrate. In this fermentation substrate a pH value of more than 7.0 will develop which provides the basis and the prerequisite for a very well functioning biocenosis. All in all enormous economic advantages can be achieved with the process described above to which the advantages of a plant production and the advantage of the production of smaller systems and the coverage of a far greater market segment can be added.

Further the following advantages can be achieved by the described device and the described process:
With the fully fermented injected mass the maximum possible amount of viable, anaerobic bacteria is introduced into the fresh substrate, i.e. a strong culture causing a violent start of the fermentation process and maintaining the strong activity for an unlimited period of time.
With the increase of the pH value to more than 7.0 which is favourable for the biocenosis an environment is provided in which the fermentation process can take place optimally and undisturbed.
Due to the fact that the reintroduction rate (the injected amount) can be accurately controlled substrates having a low pH value of less than 7.0 are enhanced via the injection amount. It may therefore well be necessary to increase the injection amount and to thus provide an environment favourable for the biocenosis in an anaerobic symbiosis from the beginning.

In general organic residues consist of the three basic components: carbohydrates, organic fats and proteins. Said three basic components play an important role in the anaerobic fermentation and contain a certain potential of energy. In scientific studies it was found that with respect to the decomposed organic dry substance (ODS) the following amounts of biogas having the corresponding quality can be extracted from the three basic components.

| | |
|---|---|
| Carbohydrates | 790 cm³/kg incl. about 50% CH₄ + about 50% CO₂ |
| yield | |
| Organic fats | 1,250 cm³/kg incl. about 68% CH₄ + about 32% CO₂ |
| Proteins | 704 cm³/kg incl. about 71% CH₄ + about 29% CO₂ |
| biogas | approx. 2,744 m³/kg decomposed ODS |

In the present fermentation process highest decomposition rates of the organic dry substance are possible, the high reintroduction rate (injected mass) substantially contributing to this. 50% of the fermenting mass will pass the process twice. But even a normal flow-through of 10 days ensures an optimum decomposition rate of the organic mass. The anaerobic process of the present invention is completed in approximately 6 to 10 days. Assuming a decomposition rate of approximately 70% of the ODS 1.920 m³ biogas per kg decomposed ODS can be obtained with this process, the biogas containing a percentage of approximately 68-74% CH₄ which will yield biogas with 7.1 kWh/m³.

It should also be mentioned that the digestion chamber load is a term adopted for fermentation from the sewage treatment technology. It only has a meaning where a stirring and mixing takes place in the reactor and is therefore irrelevant for the present process. Values of about 6.0-6.5 kg ODS/m³ digestion space will not affect the fermentation process of the present method while in processes according to the state of the art maximum values of approximately 1.5 kg ODS/m³ digestion space to 2.5 kg ODS/m³ digestion space are cited.

FIG. 4 shows a progression of the fermentation which is securely reached by the process of the present invention. The progression of the decomposition of the organic dry substance (ODS) shown in this figure is securely reached by the process of the present invention.

FIG. 5 shows a schematic cross sectional view of a heat exchanger of the device according to FIG. 2. In the central portion the housing of the heat exchanger 88 comprises a lying, double-walled portion 108 provided with terminations 110, 112 tapered towards the outside on both sides. The double-walled portion 108 is preferably cylindrical, and the left and right terminations 110, 112 are correspondingly funnel-shaped, however, alternatively other shapes of the cross section such as, for example, a rectangular cross section, are also possible. The double-walled portion 108 of the heat exchanger housing is connected to the left and right termination 110, 112 via a flange 114 with holes at both ends. The right termination 112 is provided with a tube socket 116 at its lower flank, said tube socket being connectable to the fresh substrate pump 92, for example by means of a flange with holes. The left termination 110 of the heat exchanger housing is provided with a tube socket 118 which is connectable to the mixing unit 86, for example by means of a flange with holes. The space of the double-walled portion 108 of the heat exchanger housing defined by the double wall serves as a heat carrier space 120 provided with a feed line 122 for introducing a hot fluid, preferably water, and a discharge line 124 for discharging the fluid. The fluid which can be passed through the heat carrier space 120 may, for example, be heated in a combined heat and power unit. The temperature of the fluid at the feed line 122 is preferably 80° C. The double-walled portion 108 thus forms a heat introduction body by which the organic fresh substrate supplied to the interior of the heat exchanger 88 via the tube socket 116 and dischargable via the tube socket 118 after having been pre-heated can be pre-heated. The operation of the heat exchanger 88 in combination with the temperature sensor 90 was already described in connection with FIG. 2. Furthermore three double-walled heat carrier pipes 126 which are formed so that a feed line surrounds a return line are arranged in the interior of the heat exchanger 88. Therefore the same fluid that is suppliable to the heat carrier space 120 is suppliable via an inlet 128 of the feed line, said fluid passing the heat carrier pipe 126 in the feed line from the left to the right in the outer pipe jacket and being returned to an outlet 130 of the return line from the left to the right in the return line in the central portion of the heat carrier pipe 126 at the left end of the heat carrier pipe 126. The three heat carrier pipes 126 thus also serve as a heat introduction body for pre-heating the substance present in the interior of the heat exchanger 88 to preferably 35-37° C. On the right side the heat carrier pipes 126 are fixed by the right termination 112 of the heat exchanger housing. On the left side the three heat carrier pipes 126 are fixed by a supporting disk 132.

FIG. 6 shows a schematic cross sectional view along the line I-I of FIG. 5. The supporting disk 132 comprises three fins 134 extending from the centre to the periphery and respectively provided with a centrally arranged accommodation hole 136. The accommodation holes 136 are formed as blind holes and serve to accommodate and support the left ends of the heat carrier pipes 126. A hole formed in the centre of the supporting disk 132 is also formed as a blind hole and provided with a bearing 138 so that the left end of a spindle 140 can be supported therein.

Referring to FIG. 5 again the spindle 140 extends centrally in the heat exchanger 88 in the longitudinal direction of the heat exchanger 88. The portion of the spindle 140 extending parallel to the heat carrier pipes 126 is provided with a thread. The right end of the spindle 140 is lead out of the right termination 112 of the heat exchanger housing and connected to a drive unit 142. During the operation of the heat exchanger 88 protein will flocculate from approximately 60° C. during the pre-heating of viscous biomass in the heat exchanger 88 and deposit on the surfaces of the heat introduction bodies 108, 126. For removing these deposits a cleaning device 144 is provided in the heat exchanger 88. The cleaning device 144 comprises a cleaning disk 146 the outer diameter of which is minimally smaller than the inner diameter of the double-walled portion 108. Here the term “minimally smaller” means that only so much of a tolerance is provided between the elements as required for a relative movement with respect to each other. Besides three cleaning disks 148 are provided each of which surrounds a respective heat carrier pipe 126, the outer diameter of the heat carrier pipes 126 being minimally smaller than the inner diameters of the cleaning disks 148. The outer diameter of the cleaning disks 148 and the inner diameter of the cleaning disk 146 are dimensioned so that a distance is formed between the inner diameter of the cleaning disk 146 and the outer diameter of the cleaning disks 148 as well as between the outer diameter of the cleaning disks 148 and the spindle 140 in the mounted state. The cleaning disks 146 and 148 are arranged between two cleaning support disks 150.

FIG. 7 shows a schematic cross sectional view along the line II-II in FIG. 5. The cleaning support disks have a ring-shaped portion the outer diameter of which is smaller than the inner diameter of the double-walled portion 108. From this ring-shaped portion three fins extend inwards, a thread corresponding to the thread of the spindle 140 being formed at the intersection point of the three fins in the centre of the cleaning support disk 150. Each of the three fins is provided with a ring-shaped section respectively disposed in the centre, i.e. in this section the fin surrounds a heat carrier pipe 126 in a ring-like manner in the mounted state. In this case the inner diameter of said ring-like portion of the fins is larger than the outer diameter of the heat carrier pipes 126.

Again referring to FIG. 5 the cleaning disks 146 and 148 are supported and guided by the cleaning support disks 150 configured as described above on both sides.

For removing deposits from the heat introduction bodies 108, 126 the drive unit 142 is moved in one direction (for example by an electric motor running clockwise or counter clockwise) during the operation of the heat exchanger 88 so that the entire cleaning device 144 is moved in one direction by the thread engagement between the spindle 140 and the cleaning support disk 150 since the heat carrier pipes 126 prevent a rotation of the cleaning device 144 together with the spindle 140. Thereby the cleaning disks 146 brush over the heat carrier pipes 126 and the cleaning disks 148 via the inner side of the double-walled portion 108. When reaching a restriction the drive unit 142 is switched so that the spindle 140 rotates in the opposite rotational direction. Thus the cleaning device 144 is moved in the opposite direction until a restriction is reached again. Said restriction may be a time limit, or it may be detected by means of sensors. The restriction substantially corresponds to the right and the left end of the heat carrier pipes 126, the right and the left end of the thread of the spindle 140 and the right and the left end of the double-walled portion 108. With the spindle drive a reciprocation of the cleaning device can be achieved by which the heat introduction bodies 108, 126 can be freed of deposits. The scraped-off deposits are introduced into the regular substrate flow since they are necessarily pushed into the left termination 110 of the heat exchanger housing and mixed with the substrate during each pumping cycle. Besides a continuous mixing of the fresh substrate is achieved by the reciprocating movement of the cleaning device 144 so that a uniform preheating of the fresh substrate is ensured.

The left and the right termination 110 and 112 may be removed from the double-walled portion 108 with a few hand movements so that the heat exchanger 88 can be readily checked from the inside.

As an alternative to the spindle 140 an electro-hydraulically driven cylinder or an pneumatically driven cylinder may also be provided for moving the cleaning device 144.

FIG. 8 shows a schematic representation of a further development of the device for generating biogas according to the invention. The device according to FIG. 8 differs from the device described above in that, in addition, a gasholder 152 is provided. The gasholder 152 surrounds the biogas reactor 10 so that only the lower portion 16 of the exterior housing remains exposed. The outlet of the discharge pipe 40 is slightly elongated in this further development so that the outlet can be lead through the gas container 152. In this further development the liquid reservoir 66 is displaced downwards so that the liquid reservoir 66 is disposed below the gas container 152. The biogas generated in the biogas reactor 10 is discharged from the biogas reactor and is guided to the liquid reservoir 66 via the gas discharge line 64 extending in the gas container 152. The biogas discharged from the liquid reservoir 66 is supplied to the gasholder 152. The biogas can be extracted from the gas storage 152 via a gas supply line 154.

As an alternative to the described embodiment it is possible to omit the heat exchanger 88 in the device for generating biogas shown in FIG. 2 and to pre-heat the fresh substrate by means of an introduction of steam.

The features of the invention disclosed in the above description, in the drawings as well as in the claims may be important for the realisation of the invention individually as well as in any combination.

Further the applicant expressly reserves the right to claim the following items within the framework of the present invention:

A heat exchanger for a biogas system comprising heat introduction bodies 108, 126 for heating a mass present in the heat exchanger 88 by means of a spatially separated fluid which is characterised by a cleaning device 144 which can be slipped over the heat introduction bodies 108, 126 when the heat exchanger 88 is closed.

A further developed heat exchanger characterised in that the cleaning device 144 can be reciprocated by means of a spindle drive 140.

A further developed heat exchanger characterised in that the heat introduction body 108, 126 is constituted by double-walled pipes in which the feed line surrounds the return line or the return line surrounds the feed line.

### LIST OF NUMERALS

10 biogas reactor
12 exterior housing
14 upper portion of the exterior housing
16 lower portion of the exterior housing
18 interior housing
20 charging chamber
22 overflow rim
24 lower portion of the interior reservoir
26 inner pipe
28 backflow pipe
30 first intermediate chamber
32 second intermediate chamber
34 charging orifice
36 backflow channel
38 injection pump
40 discharge pipe
42 discharge channel
44 pipe section
46 pipe section
48 slider
50 slider
52 slider
54 slider
56 receptacle
58 receptacle
60 insulation
62 heater
64 gas discharge line
66 liquid reservoir
68 gas supply line
70 pipe section
72 riser pipe
74 orifices
76 liquid
78 end of the gas discharge line
80 slider
82 slider
84 receptacle
86 mixing unit
88 heat exchanger
90 temperature sensor
92 fresh substrate pump
94 crushing unit
96 cutting tool
98 bushing
100 shaft
102 hopper
104 drive unit
106 slider
108 double-walled portion of the heat exchanger housing
110 left termination of the heat exchanger housing
112 right termination of the heat exchanger housing
114 flange with holes
116 tube socket
118 tube socket
120 heat carrier space
122 feed line
124 discharge line
126 heat carrier pipe
128 inlet
130 outlet
132 supporting disk
134 fin
136 accommodation hole
138 bearing
140 spindle
142 drive unit
144 cleaning device
146 cleaning disk
148 cleaning disk
150 cleaning support disk
152 gasholder
154 gas supply line

1. A device for generating biogas from organic materials comprising a biogas reactor provided with a charging chamber to be charged with the organic materials and a backflow channel for at least partly discharging the organic materials from the biogas reactor, characterised in that the biogas reactor further comprises at least one intermediate chamber, the charging chamber, the at least one intermediate chamber and the backflow channel forming, in the named order, parts of a flow path for the organic materials which can be passed in only one direction, one of the two portions succeeding each other respectively forming a rising flow path while the other forms a falling flow path.

2. The device according to claim 1, characterised in that the charging chamber comprises a overflow rim by means of which the at least one intermediate chamber can be charged with the organic materials from the charging chamber.

3. The device according to claim 2, characterised in that a total of at least two intermediate chambers is provided, the intermediate chambers being connected in a section below a charging orifice of the backflow channel and above the overflow rim.

4. The device according claim 3 characterised in that the intermediate chambers are formed in the interior of a cup-like interior reservoir arranged in the interior of the biogas reactor.

5. The device according, claim 1 characterised in that a discharge channel branches off the backflow channel so that the discharge channel forms a communicating pipe together with the backflow channel.

6. The device according to claim 2, characterised in that the cross sectional area of the charging chamber is narrowed in the area of the overflow rim.

7. The device according to claim 2, characterised in that the biogas reactor comprises an upper portion tapered towards the upper side and in that the overflow rim extends so far into the upper portion that the cross sectional area of the charging chamber is narrowed in the area of the overflow rim.

8. The device according to claim 2 characterised in that the biogas reactor comprises a lower portion tapered towards the bottom, a receptacle for sediments being formed in the lower section of the lower portion which, on the one hand, is optionally connectable to the interior of the biogas reactor and, on the other hand, optionally connectable to the surroundings of the biogas reactor.

9. The device according to, claim 1 characterised in that a gas discharge line is connected in the upper section of the biogas reactor via which a constant pressure can be applied to the interior of the biogas reactor.

10. The device according to claim 9, characterised in that furthermore a liquid reservoir is provided which can be charged with a liquid, the end of the gas discharge line leading out of the interior of the biogas reactor being placable in the liquid so that the pressure in the interior of the biogas reactor is adjustable via the immersion depth in the liquid.

11. The device according to, claim 10 characterised in that furthermore a mixing unit for charging the charging chamber is provided which is adjusted to mix new organic materials and organic materials discharged from the biogas reactor by means of a recirculation conduit in a ratio of substantially 1:1.

12. The device according to, claim 11 characterised in that furthermore a heat exchanger disposed upstream of the charging chamber and adjusted to pre-heat the organic materials to be newly supplied by means of a hot fluid is provided.

13. The device according to, claim 12 characterised in that a crushing unit is provided upstream of the charging chamber.

14. The device according to, claim 1 characterised in that a gasholder is provided in which biogas generated in the biogas reactor is storable and which at least partly surrounds the biogas reactor.

15. A process for generating biogas comprising the steps of:
charging a charging chamber of a biogas reactor with organic materials;
unidirectionally delivering the organic materials along the portions formed by the charging chamber, at least one intermediate chamber and a backflow channel, two subsequent portions respectively forming a rising flow path and a falling flow path; and
degassing the organic materials by means of a temporary exposure of the organic materials during the transition between the portions.

16. The process according to claim 15, characterised in that the process steps taking place inside of the biogas reactor are carried out without an active stirring of the organic materials.

17. The process according to claim 15, characterised in that the organic materials are pre-heated to 35° C.-37° C. by means of a heat exchanger before being charged into the charging chamber.

18. The process according to claim 15, characterised in that the organic materials with which the charging chamber is charged are mixed from newly supplied, pre-heated organic materials and organic materials directly discharged by means of the backflow channel.

19. The process according to claim 18, characterised in that the mixture ratio is 1:1.

20. The process according to claim 15, characterised in that an internal pressure is kept constant in the interior of the biogas reactor.

21. The process according to claim 15, characterised in that the organic materials are filled into the at least one intermediate chamber from the charging chamber via an overflow rim of the charging chamber and in that the organic materials are densified in the area the overflow rim by means of a narrowing of the cross sectional area of the charging chamber
